(19) United States
(12) Patent Application Publication
(10) Pub. No.: US 2022/0288283 A1
(43) Pub. Date: Sep. 15, 2022

(54) POLYMER FILM AND POLYMERIC BAG FOR HOLDING A MEDICAL-TECHNICAL PRODUCT TO BE IMPLANTED

(71) Applicant: Resorba Medical GmbH, Nürnberg (DE)

(72) Inventors: Michael AHLERS, Stein (DE); Markus BAUER, Nürnberg (DE); Beate RUHL, Spangenberg (DE)

(21) Appl. No.: 17/633,732

(22) PCT Filed: Aug. 26, 2020
(86) PCT No.: PCT/EP2020/073884

§ 371 Date: Feb. 8, 2022

(30) Foreign Application Priority Data
Aug. 30, 2019 (DE) 10 2019 213 178.6

(51) Int. Cl. A61L 31/04; C09D 189/00; A61L 31/16; A61L 31/14; A61L 31/00

(52) U.S. Cl. CPC A61L 31/044; C09D 189/00; A61L 31/16; A61L 31/148; A61L 31/005; A61L 2300/45; A61L 2300/406

(57) ABSTRACT

The polymer film serves for the embedding of a medical technology product to be implanted in a human organism. The polymer film includes a polymer of natural origin being biodegradable and absorbable by the human body. The polymer film has a polymer content. The polymer film further includes two antimicrobial active ingredients having a different mechanism of action and the polymer film has a total active-ingredient content. The ratio of the total active-ingredient content to the polymer content is at least 15%. The polymer content is greater than the total active-ingredient content. The polymer film is bendable and modulable and uninterrupted. The polymer forms a polymer matrix in which the antimicrobial active ingredients are embedded in a homogeneously distributed manner. The polymer film has, for each of the antimicrobial active ingredients, an individual active-ingredient content deviating from one another by at most 20%.

### CROSS-REFERENCES TO RELATED APPLICATIONS

This application claims the priority of German Patent Application, Serial No. 10 2019 213 178.6, filed Aug. 30, 2019, the content of which is incorporated herein by reference in its entirety as if fully set forth herein.

### FIELD OF THE INVENTION

The invention relates to a polymer film for the embedding of a medical technology product to be implanted in a human organism. The invention further relates to a polymer pouch for receiving of such a medical technology product, which polymer pouch has been produced from at least one such polymer film.

### BACKGROUND OF THE INVENTION

Such a medical technology product to be implanted can, for example, be knee and hip endoprostheses or a pacemaker. Every year, several hundreds of thousands of such products are implanted in the USA and in Europe. In the majority of cases, this works smoothly. However, there is an increasing number of problematic cases in which complications occur, especially because an infection occurs at the site of implantation. For the patient affected, such complications may have very serious consequences, culminating in death. Moreover, controlling the complications is associated with considerable costs.

To avoid infections when implanting such medical technology products, use is made of, inter alia, antibiotic-containing sponges and films, such as, for example, the corresponding products sold by the applicant under the brand name “GENTA-Coll”.

A further method for avoiding infection is described in U.S. Pat. No. 9,023,114 B2. According to this, the medical technology product in question is, prior to its implantation, placed in a pouch consisting of a absorbable polymer material which has been additionally provided with an infection-inhibiting active ingredient. However, said pouches are in some cases brittle and stiff and thus difficult-to-handle. Moreover, their compatibility may be limited as a consequence of the use of synthetic components.

A similar pouch is described in WO 2014/046 741 A1. Said pouch consists of an extracellular matrix, this being a cleaned piece of an animal tissue. Said matrix can be impregnated with a solution of an infection-inhibiting active ingredient. However, the then resulting adhesion of the active ingredient to the matrix is low. In particular, it can also be rinsed off. Moreover, it is consumed relatively rapidly after the insertion of the pouch into the patient's body, meaning that the infection-inhibiting action can only be in effect for a comparatively short time.

Furthermore, in the scientific paper of Chen, D. W. et al., “Sustainable release of vancomycin, gentamicin and lidocaine from novel electrospun sandwich-structured PLGA/collagen nanofibrous membranes”, International Journal of Pharmaceutics 430 (2012), pages 335-341, a membrane to be embedded into the human body having a sandwich structure is described, which consists of three sublayers produced by electrospinning and thus each consisting of nanofibers. The two outer layers are composed of a combination of the materials polylactide-polyglycolide (PLGA) and collagen, whereas the central layer contains PLGA and the three active ingredients vancomycin, gentamicin and lidocaine. Due to the production by electrospinning and the resulting composition of nanofibers, this membrane is not tight. It has fine pores.

### SUMMARY OF THE INVENTION

It is an object of the invention, then, to specify a polymer film of the type referred to at the start, having improved properties compared to the prior art.

This object is achieved by specifying a polymer film comprising, as main constituent, a polymer of natural origin that is biodegradable and is absorbable by the human body, and it has a polymer content. Moreover, it comprises, as further constituents, at least two antimicrobial active ingredients having a different mechanism of action in each case. The polymer film has in this respect a total active-ingredient content. The ratio of the total active-ingredient content to the polymer content is at least 15%, with the polymer content being, however, greater than the total active-ingredient content. Furthermore, the polymer film is bendable and modulable, so that the polymer film can be applied closely on the medical technology product, wherein the polymer film at least mostly matches itself to the contours of the medical technology product. Moreover, the polymer film is uninterrupted. Furthermore, the polymer forms a polymer matrix in which the at least two antimicrobial active ingredients are embedded in a homogeneously distributed manner. Apart from this, the polymer film has, for each of the at least two antimicrobial active ingredients, an individual active-ingredient content in each case, the individual active-ingredient contents deviating from one another by at most 20%.

The polymer film according to the invention has an exceptionally high total active-ingredient content of at least 15% and especially of at least 20%, based on the polymer content. As a result, and also because the antimicrobial active ingredients differ from one another in their particular mechanism of action, the inhibition of infection and/or suppression of pathogens is particularly efficient. Each antimicrobial active ingredient has a different mechanism of action, meaning that it can develop its action against a different pathogen class. Owing to the high total active-ingredient content and the different mechanisms of action, practically all pathogens which might possibly gather or form on the device to be implanted (=medical technology product) are killed and thus neutralized. Consequently, infections are reliably suppressed at the site of implantation. Moreover, the use of at least two antibiotic active ingredients having a different mechanism of action in each case circumvents the existing resistance of some pathogens with respect to individual active ingredients. Infections are prevented nevertheless, since at least one of the active ingredients is still effective despite the possible singular resistance of a pathogen.

The antimicrobial active-ingredient concentration introduced at the site of implantation by the polymer film into which the device to be implanted has been embedded is many times higher than can be achieved by a systemic administration of antimicrobial active ingredients. Owing to the polymer film used for embedding, the antimicrobial active ingredients are advantageously directly placed at the site at which they are required, and this is done in a very high concentration. Since the active-ingredient placement is only done at the implantation site, the total stress on the patient is relatively low, despite the locally very high active-ingredient concentration, when considered over the entire organism and is especially distinctly lower than in the case of an oral or parenteral administration. In this respect, a contribution is made to controlling the increasing antibiotic resistance of pathogens without compromising the patient's well-being at the same time. The local pathogen suppression is very high and efficient.

The at least two antimicrobial active ingredients are especially also situated inside the film material. They are thus preferably available over the entire duration of the degradation of the polymer. As a result, the infection-inhibiting action of the polymer film is maintained over a long period.

In particular, the polymer film has exactly two antimicrobial active ingredients. But also three or four antimicrobial active ingredients are possible. Preferably, not more than four antimicrobial active ingredients are present.

The polymer of natural origin is nevertheless the main constituent of the polymer film according to the invention, wherein the polymer of natural origin—like any other polymer of natural origin—has in particular an inherent moisture content. This percentage of moisture (or moisture content) is, for example, between 3% and 20%, preferably between 5% and 18%. It is part of the polymer content, which is used here, among others, to specify the polymer film This polymer content is greater than the (high) total active-ingredient content, which is especially made up of the sum of the individual active-ingredient contents of each of the at least two antimicrobial active ingredients.

Despite the very high total active-ingredient content, the polymer film can be handled and processed very easily. This is especially due to the bendability and preferably also the modulability of the polymer film. The latter means that the polymer film can be applied closely on an application object having almost any desired contours, such as one of the medical technology products to be embedded, the bendability and suppleness of the film meaning that it matches itself to the contours of the application object, or follows said contours, completely or at least as far as possible. It can practically completely cling to said almost any desired contours of the application object. This is also particularly significant because comparable existing products are, precisely, not bendable and especially also not modulable. In the case of excessively strong bending, existing comparative products can even break. Very high active-ingredient contents can lead to regional crystallization, and this can lead to a brittleness. In the case of the polymer film according to the invention, the good compatibility of the absorbable polymer of natural origin means that crystallization does not occur despite the higher active-ingredient content. It is therefore very supple. Advantageously, the polymer film according to the invention has no limit with respect to the extent of a possible bending.

The latter improves handling and processability. Moreover, use in operations is facilitated. Thus, regions of the polymer film that project beyond the product to be implanted can be bent over, with the result that the size of the unit composed of the product to be implanted and of the polymer film used for the embedding thereof is substantially determined by the product to be implanted. In this respect, the size of the incision to open the patient's body at the implantation site must also only be measured by the size of the product to be implanted. Owing to its bendability, the additionally present polymer film does not lead to any substantial enlargement of the incision opening to be introduced by the physician. By contrast, in the case of prior-art embedding products which do not exhibit comparable bendability, the size of the embedding product used for embedding instead of the size of the product to be implanted determines the size of the incision opening to be introduced by the physician, which then turns out to be larger. As a result, the patient is subjected to greater stress. In contrast, the polymer film according to the invention relieves the patient in so far as a smaller incision opening is sufficient for the implantation.

Moreover, the bendability also facilitates the embedding of the medical technology product in the polymer film according to the invention. Thus, the medical technology product can, for example, be wrapped or wrapped up with the polymer film in a conventional manner. The polymer film readily allows the folding-over that is required to this end and that is possibly also done multiple times. Preferably, the embedding can be done very closely and tightly.

The patient is further relieved in the case of the polymer film according to the invention as a result of said film being produced from or with a polymer of natural origin that is biodegradable and is absorbable by the human body. Such a polymer of natural origin, especially a highly purified one, can be degraded and absorbed more easily by the human body than the synthetic polymers used in the products of the prior art. Moreover, such a polymer is better compatible than a product composed of tissue pieces. The polymer film according to the invention has, in this respect, a higher biocompatibility than the prior art with better pathogen suppression at the same time. The absorbable polymer of natural origin is preferably an especially completely reconstituted material.

Preferably, the medical technology product to be embedded and implanted can be a pacemaker, an implantable defibrillator, a device for resynchronization therapy, an implantable medicament pump, a neurostimulation implant (spinal cord stimulator, SCS), an artificial heart, a blood pressure-regulating implant, a neuromodulator (deep brain stimulation device) or else mechanical implants, prostheses, etc. For embedding, the medical technology product in question is enclosed especially with the polymer film according to the invention, preferably as completely as possible. Remaining free in particular are only any (electrical) inward or outward lines and the connection points thereof, as are present for example in the case of pacemakers.

The polymer film is preferably free of interruptions and/or through-holes or through-openings. As a result, the medical technology product can advantageously be embedded inside the polymer film completely and especially also absolutely tightly.

Because the polymer forms a polymer matrix in which the at least two antimicrobial active ingredients are embedded in a homogeneously distributed manner, the active ingredients are equally present everywhere in the region of the implantation site. There are preferably no active ingredient-free regions, at which pathogen developments and infections might otherwise occur. Moreover, a long availability of the infection-inhibiting action is ensured as a result. The at least two antimicrobial active ingredients are also still present in a polymer which has already been partially degraded.

Because the individual active-ingredient contents of the at least two antimicrobial active ingredients deviate from one another by at most 20%, especially by at most 10%, it is ensured that the respectively different mechanisms of action of the individual active ingredients take effect practically equally and all bacteria and pathogens are suppressed and/or controlled substantially to the same extent.

What is favourable is one embodiment in which the polymer film adheres to itself. The connectability of the polymer film to itself is improved as a result. Furthermore, the medical technology product to be embedded in the polymer film is held very well in the polymer film as a result. Moreover, the external dimensions of the unit composed of the polymer film used for embedding and of the medical technology product become smaller when the overhanging regions of the polymer film are turned over and placed onto a different region of the polymer film and remain fixed in this turned-over state owing to the advantageous self-adhesion. This allows, for the physician, smallest possible incision openings on the patient's body during the operation to implant the medical technology product in question.

According to a further favourable embodiment, the polymer film is heat-sealable with itself, especially in the absence of auxiliaries and preferably in an adhesive-free manner. As a result, other products, such as, for example, pouches for receiving of one of the medical technology products to be implanted, can be produced from the polymer film in a simple manner. Advantageously, the connection point or connection line does not require another substance, which would otherwise have to be selected with respect to its biocompatibility. Complexity falls as a result. Moreover, biocompatibility remains high.

According to a further favourable embodiment, the polymer film adheres to a metallic surface. At least some of the medical technology products to be embedded have a metallic housing, on which the polymer film then clings particularly well and closely, preferably in an air-tight and/or smooth manner and especially without the polymer film producing bubbles and/or waves. Thus, what are avoided are air pockets, which could otherwise be problematic from the perspective of pathogen development. In particular, there is a continuous adhesion layer between the polymer film and the metal surface in question. This promotes a maximally stable securing, in the polymer film, of the medical technology product to be embedded, and prevents pathogen movements from or to the medical technology product, or at least considerably hampers the latter.

According to a further favourable embodiment, the polymer film is a physically tight barrier layer which prevents bacteria and/or pathogens from passing through. As a result, the risk of infection for the patient is further reduced.

According to a further favourable embodiment, the polymer film is transparent. As a result, the physician obtains an unobstructed clear view of the operation area and/or the medical technology product to be implanted. Operation errors, which may otherwise occur as a result of overlooking of a foreign body concealed behind a non-see-through film or of an insufficiently treated wound site, are thus avoided.

According to a further favourable embodiment, the individual active-ingredient contents of the at least two antimicrobial active ingredients are at least about the same size. In particular, the individual active-ingredient contents thus do not deviate from one another. This ensures that the respectively different mechanisms of action of the individual active ingredients take effect practically equally and all bacteria and pathogens are suppressed and/or controlled substantially to the same extent.

According to a further favourable embodiment, a first antimicrobial active ingredient inhibits the bacterial biosynthesis of proteins and/or the synthesis of nucleic acids. Moreover, a second antimicrobial active ingredient inhibits the bacterial synthesis of the cell wall. As a result, as many relevant bacteria and pathogens as possible are suppressed and/or controlled. Examples of a first antimicrobial active ingredient with inhibition of the bacterial biosynthesis of proteins are, in particular, aminoglycosides (e.g. amikacin, gentamicin, kanamycin, neomycin, netilmicin, streptomycin, tobramycin), chloramphenicol, fusidic acid, ketolides (e.g. cethromycin, narbomycin, telithromycin), lincosamides (e.g. clindamycin, lincomycin), lipopeptides (e.g. daptomycin), streptogramins (e.g. dalfopristin, quinupristin), macrolides (e.g. azithromycin, clarithromycin, erythromycin, roxithromycin), oxazolidinones (e.g. linezolid), tetracyclines (e.g. doxycycline, minocycline, tetracycline, oxytetracycline) and glycylcyclines (e.g. tigecycline). Examples of a first antimicrobial active ingredient with inhibition of the synthesis of nucleic acids are, in particular, gyrase inhibitors, i.e. inhibitors of DNA replication (such as first-generation fluoroquinolones (e.g. norfloxacin, enoxacin), second-generation fluoroquinolones (e.g. ciprofloxacin, ofloxacin), third-generation fluoroquinolones (e.g. levofloxacin), fourth-generation fluoroquinolones (e.g. moxifloxacin), aminocoumarins), inhibitors of bactericidal DNA damage (such as nitroimidazoles (e.g. metronidazole, tinidazole, nimorazole)), folic acid antagonists (such as sulfonamides (e.g. sulfadiazine, sulfadoxine, sulfamethoxazole, sulfasalazine), diaminopyrimidines (e.g. pyrimethamine, trimethoprim)) and ansamycins, i.e. inhibitors of bacterial RNA polymerase (such as rifamycins (e.g. rifampicin)). Examples of a second antimicrobial active ingredient with inhibition of the bacterial synthesis of the cell wall are, in particular, β-lactam antibiotics (such as carbapenems (e.g. imipenem, meropenem, ertapenem), cephalosporins, monobactams (e.g. aztreonam), penicillins, such as penicillin G, penicillin V, acylaminopenicillins (e.g. piperacillin, mezlocillin), aminopenicillins (e.g. ampicillin, amoxicillin), isoxazolylpenicillins (e.g. flucloxacillin, methicillin, oxacillin)), β-lactamase inhibitors (e.g. clavulanic acid, sulbactam, tazobactam) and sultamicillin, but also fosfomycin, glycopeptides (e.g. teicoplanin, vancomycin), polypeptides (e.g. bacitracin, colistin, gramicidin, polymyxin B, tyrothricin, teixobactin) and fosmidomycin. Depending on the particular application, it is, however, fundamentally also possible to use yet other antibacterial active ingredients.

According to a further favourable embodiment, gentamicin is a first antimicrobial active ingredient and vancomycin is a second antimicrobial active ingredient of the at least two antimicrobial active ingredients, and the polymer film has a gentamicin content and a vancomycin content. These two active ingredients can be introduced particularly well into the polymer film with a high concentration in both cases, without impairing other advantageous properties of said film, such as, in particular, bendability or bending capacity, self-adhesion, heat-sealability, adhesion to metallic surfaces, physical absence of interruptions, formation of a physically tight barrier layer and also transparency. Moreover, gentamicin and vancomycin cover practically the entire spectrum of pathogenic agents and/or pathogens that are responsible for infections in the implantation of medical technology products. In particular, these two active ingredients cover at least the following important pathogenic agents and/or pathogens: coagulase (-) Staphylococcus (e.g. Staphylococcus epidermidis), Staphylococcus aureus, methicillin-sensitive Staphylococcus aureus (MSSA), methicillin-resistant Staphylococcus aureus (MRSA), Escherichia coli, Haemophilus influenza and Moraxella catarrhalis, Enterococcus faecalis, Pseudomonas aeruginosa and Klebsiella pneumonia. Furthermore, gentamicin and vancomycin are preferably compatible with one another both physically and microbiologically. In particular, there is no negative mutual influencing of the spectra of activity. The mechanism of action of whichever is the other active ingredient remains unaffected. Furthermore, these two active ingredients have a very long storage stability or shelf life, especially of several years. In particular, both gentamicin and vancomycin can be stored for more than 3 years, without a deterioration in the mechanisms of action occurring. In comparison, this shelf life is distinctly shorter in the case of other active ingredients, for example only a few months in the case of rifampicin because of the temperature- and light-sensitivity thereof. The shelf life or stability of the active ingredients determines especially also the shelf life or stability and also the duration of usability of the polymer film per se.

According to a further favourable embodiment, the ratio of the gentamicin content to the polymer content and the ratio of the vancomycin content to the polymer content is at most 32% in both cases. What is then present for gentamicin and for vancomycin is, in both cases, a very high individual active-ingredient content with a very strong pathogen-suppression action, but with the other above-mentioned advantageous properties of the polymer film still being present. The gentamicin content and the vancomycin content can, in particular, be specified in both cases as a basis weight of the active ingredient in question from the two active ingredients. In particular, the gentamicin basis weight and the vancomycin basis weight is at most 1.8 mg/cm² in both cases, with the particular basis weight indicating the weight of the amount of active ingredient in question that is present within one cm² of the polymer film. In particular, the gentamicin content and the vancomycin content are at least approximately the same size. Both active ingredients can therefore develop their particular action on pathogenic agents/pathogens equally. The polymer content is, in particular, greater than the sum of the gentamicin content and of the vancomycin content.

According to a further favourable embodiment, the ratio of the gentamicin content to the polymer content and the ratio of the vancomycin content to the polymer content is about 18% in both cases. Preferably, the gentamicin basis weight and the vancomycin basis weight is about 1 mg/cm² in both cases. The properties of the polymer film are then particularly good.

According to a further favourable embodiment, the polymer of natural origin that is biodegradable and is absorbable by the human body is a polymer from the group of proteins, polypeptides and polysaccharides. Examples of proteins are, in particular, elastin, keratins and collagens. Examples of polypeptides are, in particular, caseins, gluten, myosin and collagen hydrolysates such as, for example, gelatin. Examples of polysaccharides are, in particular, cellulose derivatives, oxycellulose, viscose derivatives, cellulose acetate, glycosaminoglycans such as, for example, hyaluronic acid, starch, starch derivatives such as, for example, hydroxyethyl starch, chitosan, lignin-cellulose and blends of various polymers.

According to a further favourable embodiment, the polymer of natural origin that is biodegradable and is absorbable by the human body is a collagen, especially an atelocollagen, preferably an atelocollagen of equine origin, and most preferably a type 1 atelocollagen of equine origin. Such a polymer has a particularly high biocompatibility. The human body can degrade or absorb it particularly well. The collagen of equine origin is obtained especially from equine tendons.

According to a further favourable embodiment, the polymer film has a collagen basis weight from the range between 2.5 mg/cm² and 8.0 mg/cm², especially from the range between 5.0 mg/cm² and 6.2 mg/cm², and preferably of at least about 5.6 mg/cm². The result is a polymer film having particularly good properties. It is sufficiently stable and tear-resistant for a reliable handling during the operation without bringing about an excessively high input of material into the patient. Moreover, it is easily bendable and modulable. The collagen basis weight is, in this connection, especially an indication of the collagen content of the polymer film.

According to a further favourable embodiment, the polymer film is bendable by at least 120° and preferably by up to 180°. In particular, the polymer film was used to carry out bending tests with bends by about 170°, all of which proceeded positively, i.e. without breakage and destruction. Such a high bendability is unusual. It considerably improves the handling and the possible uses of the polymer film. The polymer film can, in particular, be completely bent over or turned over, i.e. bent by 180°, without a breakage of the polymer film occurring. The bending process can also be repeated multiple times, especially as often as required, without breakage or other destruction of the polymer film.

It is a further object of the invention to specify a polymer pouch of the type referred to at the start, having improved properties compared to the prior art.

The object concerning the polymer pouch is achieved by specifying a polymer pouch being produced from at least one polymer film according to the invention or at least one of the above-described favourable embodiments thereof, with two polymer films arranged on top of one another being regionally connected, especially heat-sealed, to one another on the edge side or two polymer-film sections arranged on top of one another being regionally connected, especially heat-sealed, to one another on the edge side.

Advantageously, said edge-side connection is achieved without use of other substances, especially without an adhesive.

In particular, the result is, depending on the embodiment, a mechanically firm connection point or linear connection zone. There can also be several, for example two or three or else even more, of such connection points or linear connection zones. Furthermore, the connection can preferably also be (water- and/or pathogen-)tight.

In particular, the polymer pouch can be produced from two polymer films placed over one another or preferably from a single turned-over polymer film, in both cases with an appropriate connection in the relevant edge regions. The turned-over embodiment is favoured by the good bendability of the polymer film.

The polymer pouch according to the invention is, similarly to the polymer film according to the invention, intended for receiving a medical technology product to be implanted in a human organism, especially a pacemaker, an implantable defibrillator, a device for resynchronization therapy, an implantable medicament pump, a neurostimulation implant (spinal cord stimulator, SCS), an artificial heart, a blood pressure-regulating implant, a neuromodulator (deep brain stimulation device), but also a mechanical implant, a prosthesis or the like.

Otherwise, the polymer pouch according to the invention has substantially the same favourable embodiments as the above-described polymer film. The polymer pouch according to the invention and its embodiments offer, then, substantially the same advantages which have already been described in connection with the polymer film according to the invention and the embodiments thereof.

What is favourable is a further embodiment in which the polymer pouch has an introduction opening, in the region of which two opposing polymer films or two opposing polymer-film end sections of a single polymer film, especially one turned over to form the polymer pouch, end in a non-flush manner, with the result that, in the region of the introduction opening, one polymer film projects beyond the other polymer film or one polymer-film end section projects beyond the other polymer-film end section. The overhang is, in particular, 0.5 cm to 1 cm. It facilitates the insertion of the medical technology product to be implanted.

Further features, advantages and details of the invention are revealed by the following description of examples.

### EXAMPLE 1 PRODUCTION OF A COLLAGEN FILM (AS EXAMPLE OF A POLYMER FILM) CONTAINING THE TWO ANTIMICROBIAL ACTIVE INGREDIENTS GENTAMICIN AND VANCOMYCIN

543 g of a collagen cake having a dry content of type 1 atelocollagen of equine origin of 141 g are added to 9000 ml of purified water heated to 40° C. that has been acidified beforehand with dilute acetic acid, and are transformed into a homogeneous suspension by means of a homogenizer The result is a pH of 3.5 to 4.2.

32 g of gentamicin sulfate are dissolved in 500 ml of acidified water and 21 g of vancomycin hydrochloride are dissolved in 500 ml of acidified water.

The gentamicin solution and the vancomycin solution are stirred into the collagen suspension such that a homogeneous mixture of all three components is formed.

Said homogeneous mixture is filled into flat blister moulds having a fill level of approx. 5 mm.

The blister moulds are transferred to a climatic cabinet, where the homogeneous mixture is dried for 48 hours with dried, sterile air and a drying regime with temperature levels of 35° C. to 22° C. and a humidity of 10-50% r. h.

Thereafter, the collagen films, having a collagen basis weight of about 5.6 mg/cm² and also a gentamicin basis weight and a vancomycin basis weight of about 1 mg/cm² in both cases, are removed from the blister moulds. Said collagen films are transparent, capable of adhesion (both to itself and to metallic surfaces) and bendable, in particular flexible or supple and modulable. The active ingredients gentamicin and vancomycin are distributed homogeneously in the collagen film.

### EXAMPLE 2 PRODUCTION OF A COLLAGEN POUCH (AS EXAMPLE OF A POLYMER POUCH)

Two collagen films at a time, produced as per Example 1, are conditioned in a climatic cabinet at 80% r. h., arranged in an overlapping manner, and pressed on three sides to form a collagen pouch by means of a sealing machine. This involves a pressing pressure of 8 bar and a temperature of 100° C. The pressing process lasts half a minute. During the pressing process, what occurs is a heat-sealing of the three edge regions subjected to the pressing pressure. The pressed regions are linear. There, the result are connection zones, in which there is a mechanically firm and tight connection between the two collagen films. The linear connection zones can also be referred to as sealing seams. On one side edge, there is no sealing seam present, but an introduction opening. There, the two collagen films only rest on top of one another and are, in particular, separable from one another, for example in order to insert into the collagen pouch a device to be implanted.

The pressed collagen pouch is lastly trimmed to 9×9 cm in dimension. Advantageously, only one pouch size is necessary because of the good bendability. Any empty collagen-pouch edge regions laterally projecting beyond the device to be implanted that has been inserted into the collage pouch can be readily turned over. The size of the collage pouch can thus be adapted to the conditions of the particular application without any problems.

### EXAMPLE 3 TESTING OF SEALING-SEAM STRESSABILITY

A pacemaker dummy weighing 24 g is introduced into the collagen pouch as per Example 2. The stability of the collagen pouch and of the sealing seam is tested by strong swinging and shaking of the filled collagen pouch. Both the two collagen films of the collagen pouch and the sealing seam regionally connecting the two collagen films hold out. Neither the collagen films nor the sealing seam split open.

Moreover, the collagen pouch as per Example 2 is filled with 30 ml of water and thereby to about ¾. There is no water loss within 30 min.

### EXAMPLE 4 TESTING OF FILM FLEXIBILITY—BENDING TEST

Collagen films as per Example 1 are cut into strips having a width of 1.5 cm. One such strip at a time is clamped into two opposing pairs of clamping jaws of a tensile testing machine (Zwick 2.5), with the result that the strip clamped at its two longitudinal ends is arranged between the two pairs of clamping jaws. The initial distance between the pairs of clamping jaws is 10 mm In this state, the strip running from one pair of clamping jaws to the other pair of clamping jaws is unbent.

For the test, the first pair of clamping jaws is moved to the second pair of clamping jaws at a speed of 300 mm/min until there is only a distance of 1 mm between them. After that, the first pair of clamping jaws is moved back to its initial position. This process is repeated 20 times. During the movement of the first pair of clamping jaws, the film strip clamped on both sides performs a repeated bending yielding movement in a sideward manner, wherein the closer the first pair of clamping jaws approaches the second pair of clamping jaws, the stronger the film strip bends.

During this bending test, none of the tested strips breaks. The collagen film is thus very supple. It can be highly bent without any damage. During the bending test, bends by up to about 170° were carried out.

By contrast, in the case of comparative films of a different composition, breakage of the tested strips occurred during the bending test.

### EXAMPLE 5 TESTING OF ADHESION

Collagen films as per Example 1 are cut into strips having a width of 1.5 cm and a length of more than 5 cm. A sub-region of 2 cm in length serves in each case as adhesion surface to be tested, which is also marked.

For each test, two of the film strips at a time are placed on top of one another, briefly wetted in the marked sub-regions containing the adhesion surfaces which are to be tested and are placed on top of one another (because the moistening distinctly increases again the adhesion, which is anyway already good, and thus also makes said adhesion better measurable), and pulled apart after various holding times (3-22 min) by means of the tensile testing machine (Zwick 2.5) already used in the tests as per Example 4.

Although the adhesive force increases with longer holding duration, a holding time of 10 min is chosen for the further comparative tests, since, firstly, the measurement accuracy is then already sufficiently good and, secondly, in the case of a real implantation operation as well what is realistic is a holding or preparation time of at most 10 min until the introduction of the device to be implanted, for example a pacemaker, into the tissue pouch of the patient.

In the case of the strips obtained from the collagen films as per Example 1, the average measured adhesive force is 625 mN, and this is a very good value which is close to the comparable adhesive force of a pure collagen film without any antimicrobial active ingredient. In the case of comparative films of products from other manufacturers, which likewise serve for the embedding of pacemakers to be implanted, the average measured adhesive force is distinctly lower in contrast. A comparative film has a thus ascertained adhesive force of just 20 mN. In the case of another comparative film, there is even no adhesive force at all. The ascertained value is 0 mN.

### EXAMPLE 6 TESTING OF ANTIBIOTIC ACTION

The microbiological activity of the collagen film as per Example 1 is tested in the presence of 3 Gram-positive and 3 Gram-negative pathogens. The pathogens used are Staphylococcus epidermidis, Staphylococcus aureus, Enterococcus faecalis, Escherichia coli, Pseudomonas aeruginosa and Klebsiella pneumonia. Moreover, the microbial action against a Gentamicin-resistant pathogen (Staph. aureus,>16 mg/ml) is tested.

For the tests, 3 pieces at a time, each 3×3 cm, of the collagen film are incubated with 2.5×10⁶ CFU/inoculum at 37° C. for 24 hours in accordance with ASTM E 2180 in comparison with non-microbially loaded collagen films.

The collagen film as per Example 1 exhibits a reduction of more than 4 logarithmic levels against all the pathogens tested. In accordance with the relevant definitions, an antimicrobial test is considered to be passed when a pathogen reduction of at least 99.9% (i.e. at least 3 logarithmic levels) is achieved in comparison with the reference. This assessment criterion is met in the present case. The collagen film has thus passed the antimicrobial test.

1. A polymer film for the embedding of a medical technology product to be implanted in a human organism, wherein
a) the polymer film comprises, as main constituent, a polymer of natural origin that is biodegradable and is absorbable by the human body and the polymer film has a polymer content,
b) the polymer film comprises, as further constituents, at least two antimicrobial active ingredients having a different mechanism of action in each case and the polymer film has a total active-ingredient content,
c) wherein the ratio of the total active-ingredient content to the polymer content is at least 15%, but the polymer content is greater than the total active-ingredient content,
d) the polymer film is bendable and modulable, so that the polymer film can be applied closely on the medical technology product, wherein the polymer film at least mostly matches itself to the contours of the medical technology product,
e) the polymer film is uninterrupted,
f) the polymer forms a polymer matrix in which the at least two antimicrobial active ingredients are embedded in a homogeneously distributed manner, and
g) the polymer film has, for each of the at least two antimicrobial active ingredients, an individual active-ingredient content in each case, the individual active-ingredient contents deviating from one another by at most 20%.

2. The polymer film according to claim 1, wherein the polymer film adheres to itself.

3. The polymer film according to claim 1, wherein the polymer film is heat-sealable with itself.

4. The polymer film according to claim 1, wherein the polymer film adheres to a metallic surface.

5. The polymer film according to claim 1, wherein the polymer film is a physically tight barrier layer which prevents bacteria from passing through.

6. The polymer film according to claim 1, wherein the polymer film is transparent.

7. The polymer film according to claim 1, wherein the individual active-ingredient contents of the at least two antimicrobial active ingredients are at least about the same size.

8. The polymer film according to claim 1, wherein a first antimicrobial active ingredient inhibits at least one of the bacterial biosynthesis of proteins and the synthesis of nucleic acids and a second antimicrobial active ingredient inhibits the bacterial synthesis of the cell wall.

9. The polymer film according to claim 1, wherein gentamicin is a first antimicrobial active ingredient and vancomycin is a second antimicrobial active ingredient of the at least two antimicrobial active ingredients, and the polymer film has a gentamicin content and a vancomycin content.

10. The polymer film according to claim 9, wherein the ratio of the gentamicin content to the polymer content and the ratio of the vancomycin content to the polymer content is at most 32% in both cases.

11. The polymer film according to claim 9, wherein the ratio of the gentamicin content to the polymer content and the ratio of the vancomycin content to the polymer content is about 18% in both cases.

12. The polymer film according to claim 1, wherein the polymer of natural origin that is biodegradable and is absorbable by the human body is a polymer from the group of proteins, polypeptides and polysaccharides.

13. The polymer film according to claim 1, wherein the polymer of natural origin that is biodegradable and is absorbable by the human body is a collagen.

14. The polymer film according to claim 13, wherein the polymer film has a collagen basis weight from the range between 2.5 mg/cm² and 8.0 mg/cm².

15. The polymer film according to claim 1, wherein the polymer film is bendable by at least 120°.

16. The polymer pouch for receiving a medical technology product to be implanted in a human organism, produced from at least one polymer film for the embedding of a medical technology product to be implanted in a human organism, wherein
a) the polymer film comprises, as main constituent, a polymer of natural origin that is biodegradable and is absorbable by the human body and the polymer film has a polymer content
b) the polymer film comprises, as further constituents, at least two antimicrobial active ingredients having a different mechanism of action in each case and the polymer film has a total active-ingredient content,
c) wherein the ratio of the total active-ingredient content to the polymer content is at least 15%, but the polymer content is greater than the total active-ingredient content,
d) the polymer film is bendable and modulable, so that the polymer film can be applied closely on the medical technology product, wherein the polymer film at least mostly matches itself to the contours of the medical technology product,
e) the polymer film is uninterrupted,
f) the polymer forms a polymer matrix in which the at least two antimicrobial active ingredients are embedded in a homogeneously distributed manner, and
g) the polymer film has, for each of the at least two antimicrobial active ingredients, an individual active-ingredient content in each case, the individual active-ingredient contents deviating from one another by at most 20%, with one of two polymer films arranged on top of one another being regionally connected to one another on the edge side and two polymer-film sections arranged on top of one another being regionally connected to one another on the edge side.

17. The polymer pouch according to claim 16, wherein there is an introduction opening, in the region of which one of two opposing polymer films and two opposing polymer-film end sections of a single polymer film end in a non-flush manner, with the result that, in the region of the introduction opening, one of one polymer film projects beyond the other polymer film and one polymer-film end section projects beyond the other polymer-film end section.

18. The polymer film according to claim 1, wherein the polymer of natural origin that is biodegradable and is absorbable by the human body is an atelocollagen.

19. The polymer film according to claim 1, wherein the polymer of natural origin that is biodegradable and is absorbable by the human body is an atelocollagen of equine origin.

20. The polymer film according to claim 1, wherein the polymer of natural origin that is biodegradable and is absorbable by the human body is a type 1 atelocollagen of equine origin.

21. The polymer film according to claim 13, wherein the polymer film has a collage basis weight from the range between 5.0 mg/cm² and 6.2 mg/cm²

22. The polymer film according to claim 13, wherein the polymer film has a collage basis weight of at least about 5.6 mg/cm².

23. The polymer film according to claim 1, wherein the polymer film is bendable by up to 180°.
